SEQUENCE LISTING (5 sequences)

SEQ ID NO: 1 - DNA, 20 bp, Artificial Sequence
  Synthetic construct
agagtttgatcctggctcag

SEQ ID NO: 2 - DNA, 19 bp, Artificial Sequence
  Synthetic construct
ggttaccttgttacgactt

SEQ ID NO: 3 - DNA, 1438 bp, Enterococcus faecium
gcggctggctccaaaaggttacctcaccgacttcgggtgttacaaactctcgtggtgtgacgggcggtgtgtacaaggcccgggaacgtattcaccgcggcgtgctgatccgcgattactagcgattccggcttcatgcaggcgagttgcagcctgcaatccgaactgagagaagctttaagagattagcttagcctcgcgacttcgcaactcgttgtacttcccattgtagcacgtgtgtagcccaggtcataaggggcatgatgatttgacgtcatccccaccttcctccggtttgtcaccggcagtcttgctagagtgcccaactgaatgatggcaactaacaataagggttgcgctcgttgcgggacttaacccaacatctcacgacacgagctgacgacaaccatgcaccacctgtcactttgcccccgaaggggaagctctatctctagagtggtcaaaggatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgttagctgcagcactgaagggcggaaaccctccaacacttagcactcatcgtttacggcgtggactaccagggtatctaatcctgtttgctccccacgctttcgagcctcagcgtcagttacagaccagagagccgccttcgccactggtgttcctccatatatctacgcatttcaccgctacacatggaattccactctcctcttctgcactcaagtctcccagtttccaatgaccctccccggttgagccgggggctttcacatcagacttaagaaaccgcctgcgctcgctttacgcccaataaatccggacaacgcttgccacctacgtattaccgcggctgctggcacgtagttagccgtggctttctggttagataccgtcaagggatgaacagttactctcatccttgttcttctctaacaacagagttttacgatccgaaaaccttcttcactcacgcggcgttgctcggtcagactttcgtccattgccgaagattccctactgctgcctcccgtaggagtttgggccgtgtctcagtcccaatgtggccgatcaccctctcaggtcggctatgcatcgtggccttggtgagccgttacctcaccaactagctaatgcaccgcgggtccatccatcagcgacacccgaaagcgcctttcaaatcaaaaccatgcggttttgattgttatacggtattagcacctgtttccaagtgttatccccttctgatgggcaggttacccacgtgttactcacccgttcgccactcctctttttccggtggagcaagctccggtggaaaaagaagcgtgcgacttgcacgtattaggc

SEQ ID NO: 4 - DNA, 1487 bp, Enterococcus azikeevi
agagtttgaatcatggctcaggacgaacgctggcggcgtgcctaatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggcgacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcatttagttgggcactctagcaagactgccggtgacaaaccggaggaaggtggggatgacgtcaaatcatcatgccccttatgacctgggctacacacgtgctacaatgggaagtacaacgagtcgcaaagtcgcgaggctaagctaatctcttaaagcttctctcagttcggattgtaggctgcaactcgcctacatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgat

SEQ ID NO: 5 - DNA, 1429 bp, Enterobacter roggenkampii
gcagctacacatgcaagtcgagcggcagcggaagtagcttgctactttgccggcgagcggcggacgggtgagtaatgtctgggaaactgcctgatggagggggataactactggaaacggtagctaataccgcataacgtcgcaagaccaaagagggggaccttcgggcctcttgccatcagatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgttgaggttaataacctcagcgattgacgttacccgcagaagaagcaccggctaactccgtgccagcagccgcggtaatacggagggtgcaagcgttaatcggaattactgggcgtaaagcgcacgcaggcggtctgtcaagtcggatgtgaaatccccgggctcaacctgggaactgcattcgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgctaccacttgatt

(19) United States
(12) Patent Application Publication
(10) Pub. No.: US 2021/0221720 A1
(43) Pub. Date: Jul. 22, 2021

(54) PATHOGENIC BACTERIA AND METHOD FOR DEGRADING ETHYLENE OXIDE

(71) Applicants: Chio Kang Medical, Inc., Palo Alto, CA (US); QIAOKANG BIOTECH (GUANGDONG) CO., LTD., Guangzhou City (CN)

(72) Inventors: Jianlong Xue, Guangzhou City (CN); Dongxin Hou, Guangzhou City (CN); Yecheng He, Guangzhou City (CN); Xuzhong Liao, Guangzhou City (CN); Shengwei Hu, Guangzhou City (CN); Xin Yin, Guangzhou City (CN); Qinghua Xiao, Guangzhou City (CN); Liqing Zhu, Guangzhou City (CN); Jiali Lin, Guangzhou City (CN); Yuhua Zou, Guangzhou City (CN); Lixiong Feng, Guangzhou City (CN)

(21) Appl. No.: 17/012,828

(22) Filed: Sep. 4, 2020

Related U.S. Application Data
(63) Continuation of application No. PCT/CN2020/101144, filed on Jul. 9, 2020.

(30) Foreign Application Priority Data
Jan. 20, 2020 (CN) 202010062876.4
Jan. 20, 2020 (CN) 202010064633.4
Jan. 20, 2020 (CN) 202010064716.3
Jan. 20, 2020 (CN) 202010065460.8

(51) Int. Cl. C02F 3/34; C12N 1/20

(52) U.S. Cl. CPC C02F 3/345; C12R 1/01; C12N 1/20

(57) ABSTRACT

The present disclosure discloses three pathogenic bacteria (i.e., Enterococcus faecium EO-04, Enterococcus azikeevi EO-07, and Enterobacter roggenkampii EO-10) capable of degrading ethylene oxide and a method for degrading ethylene oxide. The pathogenic bacteria are conserved in China General Microbiological Culture Collection Center, under deposit numbers CGMCC No.18434, CGMCC No.18437, CGMCC No.18440, respectively.

### CROSS-REFERENCE TO RELATED PATENT APPLICATIONS

This application is a Bypass Continuation of PCT/CN2020/101144, filed Jul. 9, 2020, which application claims the benefit of Chinese Patent Application No. 202010062876.4, filed on Jan. 20, 2020, Chinese Patent Application No. 202010064716.3, filed on Jan. 20, 2020, Chinese Patent Application No. 202010065460.8, filed on Jan. 20, 2020 and Chinese Patent Application No. 202010064633.4, filed on Jan. 20, 2020, the entire contents of which are incorporated herein by reference in their entirety for all purposes.

### TECHNICAL FIELD

The present disclosure relates to the technical field of biodegradation, and more particularly relates to a pathogenic bacteria and a method for degrading ethylene oxide using the same.

### SEQUENCE STATEMENT

Incorporated by reference herein in its entirety is the Sequence Listing entitled “1211_CK04_ST25” created, Jun. 17, 2020, size of 6.6 kilobytes.

### BACKGROUND

Ethylene oxide (EO), one of important petrochemicals in modern chemical industry, is a broad-spectrum and efficient bactericidal disinfectant. As ethylene oxide can kill most of bacteria, spores, viruses, and fungi, and has a strong penetrability to a deep layer of articles, EO has an irreplaceable role in medical sterilization. However, ethylene oxide is extremely active, flammable and combustible, and is a well-known carcinogen, which limits the applications of ethylene oxide.

Currently, there primarily are two methods for treating ethylene oxide waste gas in industry. One method is to use sulfuric acid to neutralize EO. However, this method has a low absorption saturation and a low treatment efficiency, and may result in undesirable side products and thus increase treatment cost. The other method is to perform oxidation reaction in an oxidation reactor. In the method, a high-level control of technical parameters is required, and explosion is prone to occur.

Therefore, there is an urgent need to provide a safe and efficient method for the treatment of ethylene oxide waste gas.

### SUMMARY

In view of this, the present disclosure provides Enterococcus faecium, Enterococcus azikeevi, and Enterobacter roggenkampii strains that can effectively degrade ethylene oxide, which can be used to degrade ethylene oxide pollutants, e.g., in sewage, sludge, exhaust gas, or wastewater, especially industrial (such as industries related to petroleum and derivative products), medical treatment (such as ethylene oxide sterilant) and other sewage or wastewater. Therefore, it may greatly improve the decontamination processes of ethylene oxide and reduce environmental risks, such as public health risk. According to various embodiments, a pathogenic bacteria for degrading ethylene oxide and a method for degrading ethylene oxide are provided.

A pathogenic bacteria for degrading ethylene oxide is provided, which is selected from the group consisting of:

Enterococcus faecium EO-04, with the deposit number CGMCC No.18434;

Enterococcus azikeevi EO-07, with the deposit number CGMCC No.18437; and

Enterobacter roggenkampii EO-10, with the deposit number CGMCC No.18440.

In one of the aspects of the present disclosure, an Enterococcus faecium strain bacterium comprising the 16S rDNA sequence of SEQ ID NO: 3; an Enterococcus azikeevi strain bacterium comprising the 16S rDNA sequence of SEQ ID NO: 4; or the Enterobacter roggenkampii strain bacterium comprising the 16S rDNA sequence of SEQ ID NO: 5 is provided. These strains can effectively degrade ethylene oxide.

In one of the aspects of the present invention, a degradation agent for degrading ethylene oxide, comprising one or more strains selected from the group consisting of an Enterococcus faecium EO-04, with the deposit number CGMCC No.18434; an Enterococcus azikeevi EO-07, with the deposit number CGMCC No.18437; an Enterobacter roggenkampii EO-10, with the deposit number CGMCC No.18440; an Enterococcus faecium strain bacterium comprising the 16S rDNA sequence of SEQ ID NO: 3; an Enterococcus azikeevi strain bacterium comprising the 16S rDNA sequence of SEQ ID NO: 4; and the Enterobacter roggenkampii strain bacterium comprising the 16S rDNA sequence of SEQ ID NO: 5 is provided.

In one of the aspects, the degradation agent is prepared by culturing one or more strains.

In one of the aspects, the degradation agent has a final concentration of one or more strains in the degradation agent of at least 10¹⁰ cfu/mL.

In one of the aspects, the present disclosure provides a method for preparing a degradation agent for degrading ethylene oxide, comprising: incubating one or more strains selected from the group consisting of an Enterococcus faecium EO-04, with the deposit number CGMCC No.18434; an Enterococcus azikeevi EO-07, with the deposit number CGMCC No.18437; and an Enterobacter roggenkampii EO-10, with the deposit number CGMCC No.18440 in a liquid Sabouraud medium and at a temperature of 20-40° C.

In some aspects, the liquid Sabouraud medium comprises: by mass, 40 parts of glucose, 5 parts of casein trypsin digest, and 5 parts of animal tissue pepsin digest, which are combined in water, adjusted to a pH of 5.4-5.8, and the volume brought to 1000 parts with water.

In one of the aspects, the present disclosure provides a method for manufacturing bacteria for degrading ethylene oxide, comprising: incubating one or more strains selected from the group consisting of an Enterococcus faecium EO-04, with the deposit number CGMCC No.18434; an Enterococcus azikeevi EO-07, with the deposit number CGMCC No.18437; an Enterobacter roggenkampii EO-10, with the deposit number CGMCC No.18440; an Enterococcus faecium strain bacterium comprising the 16S rDNA sequence of SEQ ID NO: 3; an Enterococcus azikeevi strain bacterium comprising the 16S rDNA sequence of SEQ ID NO: 4; and the Enterobacter roggenkampii strain bacterium comprising the 16S rDNA sequence of SEQ ID NO: 5 in a liquid Sabouraud medium and at a temperature of 20-40° C.

In some aspects, the liquid Sabouraud medium comprises: by mass, 40 parts of glucose, 5 parts of casein trypsin digest, and 5 parts of animal tissue pepsin digest, which are well mixed in water, adjusted to a pH of 5.4-5.8, and the volume brought to 1000 parts with water.

In one of the aspects of the present invention, a method for biodegrading ethylene oxide is provided, comprising:

degrading ethylene oxide with one or more strains selected from the group consisting of an Enterococcus faecium EO-04, with the deposit number CGMCC No.18434; an Enterococcus azikeevi EO-07, with the deposit number CGMCC No.18437; and an Enterobacter roggenkampii EO-10, with the deposit number CGMCC No.18440; an Enterococcus faecium strain bacterium comprising the 16S rDNA sequence of SEQ ID NO: 3; an Enterococcus azikeevi strain bacterium comprising the 16S rDNA sequence of SEQ ID NO: 4; and the Enterobacter roggenkampii strain bacterium comprising the 16S rDNA sequence of SEQ ID NO: 5, or a degradation agent according to the invention or a degradation agent prepared according to a method of the invention.

In some aspects, the method is used to degrade ethylene oxide in waste gas or waste water and comprises mixing the waste gas or waste water with one or more strains selected from the group consisting of an Enterococcus faecium EO-04, with the deposit number CGMCC No.18434; an Enterococcus azikeevi EO-07, with the deposit number CGMCC No.18437; an Enterobacter roggenkampii EO-10, with the deposit number CGMCC No.18440; an Enterococcus faecium strain bacterium comprising the 16S rDNA sequence of SEQ ID NO: 3; an Enterococcus azikeevi strain bacterium comprising the 16S rDNA sequence of SEQ ID NO: 4; and the Enterobacter roggenkampii strain bacterium comprising the 16S rDNA sequence of SEQ ID NO: 5; or a degradation agent according to the invention or a degradation agent prepared according to a method of the invention.

In some aspects, the degrading ethylene oxide with one or more strains comprises incubating tone or more strains in a liquid Sabouraud medium and at a temperature of 20-40° C.

In some aspects, the degradation rate is at least 10% greater relative to the degradation rate of ethylene oxide in the absence of the strain for degrading ethylene oxide.

In some aspects, the method comprises the concentration of the strain for degrading ethylene oxide ranges from 10¹⁰ cfu/mL to 10¹² cfu/mL.

In one of the aspects of the present invention, a method for decreasing the amount of ethylene oxide in sample is provided, the method comprising adding to a sample comprising ethylene oxide an amount a pure culture of an Enterococcus faecium, Enterococcus azikeevi, or Enterobacter roggenkampii strain bacterium, allowing the bacterium to degrade the ethylene oxide, thereby decreasing the amount of ethylene oxide,
wherein the 16S rDNA sequence of the Enterococcus faecium strain bacterium is SEQ ID NO: 3; the 16S rDNA sequence of the Enterococcus azikeevi strain bacterium is SEQ ID NO: 4; or the 16S rDNA sequence of the Enterobacter roggenkampii strain bacterium is SEQ ID NO: 5.

In some aspects, the Enterococcus faecium, Enterococcus azikeevi, or Enterobacter roggenkampii strain bacterium is capable of using ethylene oxide as a carbon source and is capable of growing normally with ethylene oxide as the sole carbon source in the culture.

In some aspects, the Enterococcus faecium strain bacterium is Enterococcus faecium EO-04, with the deposit number CGMCC No.18434; the Enterococcus azikeevi strain bacterium is Enterococcus azikeevi EO-07, with the deposit number CGMCC No.18437; and the Enterobacter roggenkampii strain bacterium is Enterobacter roggenkampii EO-10, with the deposit number CGMCC No.18440.

In one of the aspects of the present invention, a use is provided, the use being use of one or more strains selected from the group consisting of an Enterococcus faecium EO-04, with the deposit number CGMCC No.18434; an Enterococcus azikeevi EO-07, with the deposit number CGMCC No.18437; and an Enterobacter roggenkampii EO-10, with the deposit number CGMCC No.18440; an Enterococcus faecium strain bacterium comprising the 16S rDNA sequence of SEQ ID NO: 3; an Enterococcus azikeevi strain bacterium comprising the 16S rDNA sequence of SEQ ID NO: 4; and the Enterobacter roggenkampii strain bacterium comprising the 16S rDNA sequence of SEQ ID NO: 5, or a degradation agent according to the invention or a degradation agent prepared according to a method of the invention.

In one of the aspects of the present invention, a use is provided, the use being use of one or more strains selected from the group consisting of an Enterococcus faecium EO-04, with the deposit number CGMCC No.18434; an Enterococcus azikeevi EO-07, with the deposit number CGMCC No.18437; an Enterobacter roggenkampii EO-10, with the deposit number CGMCC No.18440; an Enterococcus faecium strain bacterium comprising the 16S rDNA sequence of SEQ ID NO: 3; an Enterococcus azikeevi strain bacterium comprising the 16S rDNA sequence of SEQ ID NO: 4; and the Enterobacter roggenkampii strain bacterium comprising the 16S rDNA sequence of SEQ ID NO: 5, in preparation of a degradation agent for degrading ethylene oxide.

In one of the aspects of the present invention, a method of performing ethylene oxide tolerance and degradation acclimation to bacteria with ethylene oxide degradation potential to prepare bacteria strain having ethylene oxide tolerance and degradation ability is provided, the method comprising:

inducted acclimation for ethylene oxide tolerance, comprising: successively passaging the bacteria with ethylene oxide degradation potential by streaking the same on a series of acclimation medium for ethylene oxide tolerance containing a gradient of increasing ethylene oxide concentrations from 100 to 800 mg/L; after each passaging, incubating at 20-40° C. for 24 to 48 hours, and selecting a single colony with a largest radius for next passaging; and finally selecting a single colony with a largest colony radius on an acclimation medium containing ethylene oxide of 500-800 mg/L to obtain a bacteria strain of ethylene oxide tolerance; and

inducted acclimation for ethylene oxide degradation ability, comprising: successively passaging the bacteria strain of ethylene oxide tolerance by steaking the same on a series of acclimation medium for ethylene oxide degradation containing ethylene oxide of 500-800 mg/L and a gradient of decreasing proportion of carbon source from 50% to 0%; after each passaging, incubating at 20-40° C. for 24 to 48 hours, and selecting a single colony with a largest radius for next passaging; and finally selecting a single colony with a largest colony radius on the acclimation medium containing 500-800 mg/L of ethylene oxide and 0% of carbon source to obtain the bacteria strain having ethylene oxide tolerance and degradation ability;

wherein the series of acclimation medium for ethylene oxide tolerance have ethylene oxide concentrations increasing between 100 and 800 mg/L and comprises, by mass, 10 parts of peptone, 40 parts of glucose, and 15 parts of agar, which are mixed with water, adjusted to a pH of 5.4-5.8, and the volume brought to 1000 parts with water;

and wherein the series of acclimation medium for ethylene oxide degradation have an ethylene oxide concentration of 500-800 mg/L and comprises, by mass, 10 parts of peptone, glucose decreasing from 20 parts to 0 parts, and agar 15 parts, which are mixed with water, adjusted to a pH of 5.4-5.8, and the volume brought to 1000 parts with water.

In one of the aspects of the present invention, a method for screening and purifying bacteria with potential of ethylene oxide degradation is provided, the method comprising:

collecting microbial active sludge mixture containing ethylene oxide;

mixing the sludge mixture with phosphate buffer, clarifying and filtering to obtain a suspension;

incubating the suspension in an enriched medium containing ethylene oxide at a temperature of 20-40° C., to obtain a bacterial suspension capable of surviving in an environment containing ethylene oxide; and

incubating the bacterial suspension in a screening and purification medium containing ethylene oxide at a temperature of 20-40° C. to obtain the bacteria with potential of ethylene oxide degradation;

wherein, the enriched medium containing ethylene oxide has an ethylene oxide concentration of 100 mg/L and comprises, by mass, 40 parts of glucose, 5 parts of casein trypsin digest, and 5 parts of animal tissue pepsin digest, which are mixed with water, adjusted to a pH of 5.4-5.8, and the volume brought to 1000 parts with water;

and wherein the screening and purification medium containing ethylene oxide has an ethylene oxide concentration of 100 mg/L and comprises, by mass, 40 parts of glucose, 5 parts of casein trypsin digest, 5 parts of animal tissue pepsin digest and 15 parts of agar, which are mixed with water, adjusted to a pH of 5.4-5.8, and the volume brought to 1000 parts with water.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-1C show the Gram staining result of the EO-degrading potential bacteria, wherein the EO-degrading potential bacteria were, FIG. 1A, Enterococcus faecium EO-04 original strain, FIG. 1B Enterococcus azikeevi EO-07 original strain and FIG. 1C Enterobacter roggenkampii EO-10 original strain obtained by the enrichment, purification and screening processes according to one embodiment of the present disclosure;

FIGS. 2A-2C show bacterial colony growth of the EO-degrading potential bacteria in the enrichment medium B after growing for 48 hours at a constant temperature of 37° C., wherein the EO-degrading potential bacteria were FIG. 2A, Enterococcus faecium EO-04 original strain; FIG. 2B, Enterococcus azikeevi EO-07 original strain and FIG. 2C Enterobacter roggenkampii EO-10 original strain obtained by the enrichment, purification and screening processes according to one embodiment of the present disclosure;

FIGS. 3A-3C show the phylogenetic evolution diagram of the EO-degrading potential bacteria, wherein the EO-degrading potential bacteria were FIG. 3A, Enterococcus faecium EO-04 original strain; FIG. 3B, Enterococcus azikeevi EO-07 original strain and FIG. 3C Enterobacter roggenkampii EO-10 original strain obtained by the enrichment, purification and screening processes according to one embodiment of the present disclosure;

FIGS. 4A-4B show bacterial colony growth of Enterococcus faecium EO-04 strain (FIG. 4A) after inducted acclimation, and (FIG. 4B) before inducted acclimation in a liquid medium with 800 mg/L ethylene oxide after growing at a constant temperature of 37° C. for 48 hours in the comparative ethylene oxide degradation test, wherein the EO-04 strain was obtained by the inductive acclimation process according to one embodiment of the present disclosure.

FIGS. 5A-5B show bacterial colony growth of Enterococcus azikeevi EO-07 strain (FIG. 5A) after inducted acclimation, and (FIG. 5B) before inducted acclimation in a liquid medium with 800 mg/L ethylene oxide after growing at a constant temperature of 37° C. for 48 hours in the comparative ethylene oxide degradation test, wherein the EO-07 strain was obtained by the inductive acclimation process according to one embodiment of the present disclosure.

FIGS. 6A-6B show bacterial colony growth of Enterobacter roggenkampii EO-10 strain (FIG. 6A) after inducted acclimation, and (FIG. 6B) before inducted acclimation in a liquid medium with 800 mg/L ethylene oxide after growing at a constant temperature of 37° C. for 48 hours in the comparative ethylene oxide degradation test, wherein the EO-10 strain was obtained by the inductive acclimation process according to one embodiment of the present disclosure.

### DETAILED DESCRIPTION OF THE EMBODIMENTS

In order to further clarify the objectives, technical solutions, and advantages of the present disclosure, hereinafter the present disclosure is further explained in details in combination with the figures and specific embodiments. It should be appreciated that the specific embodiments described herein is used only to explain the present disclosure, but in no way to limit the scope of the present disclosure.

Unless otherwise defined, all the technical and scientific terms used in this specification have the same meaning as commonly understood by those skilled in the art of the technical field to which the present disclosure pertains. The terms used in the description of the present disclosure is provided only for describing specific embodiments, but not for limiting the present disclosure. The term “and/or” as used herein encompasses any or all the combinations of one or more related items listed.

The chemicals in the following specific examples of the present disclosure are all commercially available, and the methods not described are conventional experimental methods, which will not be elaborated herein.

According to one embodiment, a pathogenic bacteria for degrading ethylene oxide is provided, which is selected from the group consisting of: Enterococcus faecium EO-04, Enterococcus azikeevi EO-07, and Enterobacter roggenkampii EO-10.

Enterococcus faecium EO-04 is conserved in China General Microbiological Culture Collection Center (CGMCC, Address: NO.1 West Beichen Road, Chaoyang District, Beijing, China, Institute of Microbiology Chinese Academy of Sciences) on Aug. 29, 2019, with the deposit number CGMCC No.18434.

Enterococcus azikeevi EO-07 is conserved in China General Microbiological Culture Collection Center (CGMCC, Address: NO.1 West Beichen Road, Chaoyang District, Beijing, China, Institute of Microbiology Chinese Academy of Sciences) on Aug. 29, 2019, with the deposit number CGMCC No.18437.

Enterobacter roggenkampii EO-10 is conserved in China General Microbiological Culture Collection Center (CGMCC, Address: NO.1 West Beichen Road, Chaoyang District, Beijing, China, Institute of Microbiology Chinese Academy of Sciences) on Aug. 29, 2019, with the deposit number CGMCC No.18440.

According to one embodiment, a method for screening a pathogenic bacteria having potential to degrade ethylene oxide is provided, which includes the following steps:

(1.1) adding a waste water or sludge sample collected from a drain outlet of a sewage treatment plant or chemical plant into an enrichment medium containing ethylene oxide (e.g. 100 mg/L) for intolerance enrichment to obtain a bacteria suspension; and

(1.2) inoculating the bacteria suspension of Step (1.1) into a screening purification medium containing ethylene oxide (e.g. 100 mg/L) for screening and purifying original strains having potential to degrade ethylene oxide.

According to one embodiment, a method of preparing the enrichment medium includes the following steps:

Weighing 40 g of glucose, 10 g of casein trypsin digest, pepsin digest of animal tissue equal mixture, and 5 g of animal tissue pepsin digest, adjusting pH to 5.4-5.8; adding distilled water to 1000 mL, and distributing into 250 mL; sterilizing at 121° C. for 20 min; cooling down to room temperature; adding 25 mg of liquid ethylene oxide with a sealed injection needle; and finally obtaining an enrichment medium containing 100 mg/L of ethylene oxide.

According to one embodiment, a method of preparing the screening purification medium includes the following steps:

Weighing 40 g of glucose, 10 g of casein trypsin digest, pepsin digest of animal tissue equal mixture, 15 g of agar, adjusting pH to 5.4-5.8; adding distilled water to 1000 mL, and distributing into 250 mL; sterilizing at 121° C. for 20 min; cooling down to 50-55° C.; adding 25 mg of liquid ethylene oxide with a sealed injection needle; and finally obtaining an screening purification medium plate containing 100 mg/L of ethylene oxide.

According to one embodiment, the present disclosure provides a method for acclimatizing a pathogenic bacteria capable of degrading ethylene oxide, which includes a step of ethylene oxide tolerance acclimatization and a step of ethylene oxide degradation acclimatization.

(2.1) Ethylene oxide tolerance acclimatization: The original strains having potential to degrade ethylene oxide of Step (1.2) are streak-inoculated on ethylene oxide tolerance acclimatization medium plates containing ethylene oxide at gradually increasing concentrations, respectively, and placed in an incubator at 37° C. for 24-48 h. Finally the single colony having the greatest colony radius is selected on the tolerance acclimatization medium plate containing 500-800 mg/L of ethylene oxide to obtain an ethylene oxide-degradation dominant strain.

According to one embodiment, the present disclosure provides a method for acclimatizing pathogenic bacteria capable of degrading ethylene oxide, which includes, for example, the following steps: streak-inoculating the original strains having potential to degrade ethylene oxide of Step (1.2) on an ethylene oxide tolerance acclimatization medium plate containing 100 mg/L of ethylene oxide, incubating in an incubator at 37° C. for 24-48 h, selecting the single colony having the greatest colony radius on the plate for further streak-inoculation on an ethylene oxide tolerance acclimatization medium plate containing 100-200 mg/L of ethylene oxide, incubating in an incubator at 37° C. for 24-48 h, selecting the single colony having the greatest colony radius on the plate for further streak-inoculation on an ethylene oxide tolerance acclimatization medium plate containing 200-500 mg/L of ethylene oxide, incubating in an incubator at 37° C. for 24-48 h, selecting the single colony having the greatest colony radius on the plate for further streak-inoculation on an ethylene oxide tolerance acclimatization medium plate containing 500-800 mg/L of ethylene oxide, incubating in an incubator at 37° C. for 24-48 h, and finally selecting the single colony having the greatest colony radius on the tolerance acclimatization medium plate containing 500-800 mg/L of ethylene oxide to obtain an ethylene oxide-degradation dominant strain.

According to one embodiment, the ethylene oxide tolerance acclimatization medium may for example be: 10 g/1 of peptone, 40 g/1 of glucose, 15 g/1 of agar, 100-800 mg/L of ethylene oxide, pH 5.4-5.8.

According to one embodiment, the ethylene oxide tolerance acclimatization medium is prepared as follows:

Weighing 10 parts by weight of peptone, 40 parts of glucose, and 15 parts of agar, adjusting the pH to 5.4-5.8, adding 1000 parts of distilled water, performing sterilization, adding liquid ethylene oxide by using a sealed injection needle to obtain the tolerance acclimatization medium plates containing 100-800 mg/L of ethylene oxide.

According to one embodiment, the ethylene oxide tolerance acclimatization medium is prepared as follows:

Weighing 10 g by weight of peptone, 40 g of glucose, and 15 g of agar, adjusting the pH to 5.4-5.8, adding distilled water to 1000 mL, distributing into 250 mL, and performing sterilization at 121° C. for 20 min. The medium is heated to melt before use. When the temperature of the medium is cooled down to 50-56° C., 25-200 mg of liquid ethylene oxide is added by using a sealed injection needle to obtain the tolerance acclimatization medium plates containing 100-800 mg/L of ethylene oxide.

According to one embodiment, the ethylene oxide tolerance acclimatization medium may contain ethylene oxide with a concentration of, for example, 100-800 mg/L, for example, 100 mg/L, 200 mg/L, 300 mg/L, 400 mg/L, 500 mg/L, 600 mg/L, 700 mg/L, 800 mg/L, or any value therebetween.

(2.2) Ethylene oxide degradation acclimatization: The ethylene oxide-degradation dominant strain obtained in Step (2.1) is inoculated on ethylene oxide degradation acclimatization medium plates containing for example 800 mg/L of ethylene oxide and carbon source at gradually decreasing concentrations, respectively, incubated in an incubator at 37° C. for 24-48 h, and finally the single colony having the greatest colony radius is selected on the ethylene oxide degradation acclimatization medium plate containing 800 mg/L of ethylene oxide and 0% of carbon source to obtain a bacteria strain capable of degrading ethylene oxide.

According to one embodiment, the ethylene oxide-degradation dominant strain obtained in Step (2.1) may for example be inoculated on an ethylene oxide degradation acclimatization medium plate containing 800 mg/L of ethylene oxide and 50% of carbon source, incubated in an incubator at 37° C. for 24-48 h, the single colony having the greatest colony radius is selected on the plate for further inoculation on an ethylene oxide degradation acclimatization medium plate containing 800 mg/L of ethylene oxide and 30% of carbon source, incubated in an incubator at 37° C. for 24-48 h, the single colony having the greatest colony radius is selected on the plate for further inoculation on an ethylene oxide degradation acclimatization medium plate containing 800 mg/L of ethylene oxide and 10% of carbon source, incubated in an incubator at 37° C. for 24-48 h, the single colony having the greatest colony radius is selected on the plate for further inoculation on an ethylene oxide degradation acclimatization medium plate containing 800 mg/L of ethylene oxide and 0% of carbon source, incubated in an incubator at 37° C. for 24-48 h, and finally the single colony having the greatest colony radius is selected on the ethylene oxide degradation acclimatization medium plate containing 800 mg/L of ethylene oxide and 0% of carbon source to obtain a bacteria strain capable of degrading ethylene oxide.

According to one embodiment, the ethylene oxide degradation acclimatization medium may for example be: 10 g/L of peptone, 0-20 g/L of glucose, 15 g/L of agar, 800 mg/L of ethylene oxide, pH 5.4-5.8.

According to one embodiment, the ethylene oxide degradation acclimatization medium is prepared as follows:

Weighing 10 parts by weight of peptone, 0-20 parts of glucose, and 15 parts of agar, adjusting the pH to 5.4-5.8, adding 1000 parts of distilled water, performing sterilization, adding liquid ethylene oxide by using a sealed injection needle to obtain the tolerance degradation medium plates containing 0-50% of carbon source and 800 mg/L of ethylene oxide.

According to one embodiment, the ethylene oxide degradation acclimatization medium is prepared as follows:

Weighing 10 g of peptone, 0-20 g of glucose, and 15 g of agar, adjusting the pH to 5.4-5.8, adding distilled water to 1000 mL, distributing into 250 mL, and performing sterilization at 121° C. for 20 min. The medium is heated to melt before use. When the temperature of the medium is cooled down to 50-56° C., 200 mg of liquid ethylene oxide is added by using a sealed injection needle to obtain the degradation acclimatization medium plates containing 0-50% of carbon source and 800 mg/L of ethylene oxide.

According to one embodiment, the ethylene oxide degradation acclimatization medium may contain glucose with a concentration of 0-20 g/L, for example 1 g/L, 2 g/L, 3 g/L, 4 g/L, 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, or 20 g/L. For example, in the ethylene oxide degradation acclimatization medium, the glucose concentrations of 20 g/L, 12 g/L, 4 g/L, and 0 g/L correspond to 50%, 30%, 10%, and 0% of carbon source, respectively.

According to one embodiment, the three pathogenic bacteria capable of degrading ethylene oxide provided by the present disclosure are obtained by the method mentioned above.

According to one embodiment, the present disclosure further provides a use of the three pathogenic bacteria for the treatment of ethylene oxide.

The ethylene oxide-degrading pathogenic bacteria of the present disclosure may have the following effect:

After the acclimatization of the original strains, Enterococcus faecium EO-04 obtains the tolerance and degradation ability to ethylene oxide with a high concentration. Under a carbon-free condition, it has a degradation rate of 67.26% for 400 mg/L of ethylene oxide, and a degradation rate of 51.45% for 800 mg/L of ethylene oxide.

After the acclimatization of the original strains, Enterococcus azikeevi EO-07 obtains the tolerance and degradation ability to ethylene oxide with a high concentration. Under a carbon-free condition, it has a degradation rate of 65.42% for 400 mg/L of ethylene oxide, and a degradation rate of 50.22% for 800 mg/L of ethylene oxide.

After the acclimatization of the original strains, Enterobacter roggenkampii EO-10 obtains the tolerance and degradation ability to ethylene oxide with a high concentration. Under a carbon-free condition, it has a degradation rate of 83.75% for 400 mg/L of ethylene oxide, and a degradation rate of 53.44% for 800 mg/L of ethylene oxide.

Enrichment, Screening, and Identification of Pathogenic Bacteria for Degrading Ethylene Oxide

(1) Enrichment and screening.

1. Sample source: The samples of the embodiments are taken from a sludge mixture at the drain outlet of a sewage treatment plant in a suburb of Guangzhou, Guangdong Province.

2. Preparation of enrichment medium A: 40 g of glucose, 5 g of casein trypsin digest, 5 g of pepsin digest of animal tissue were weighed. The pH was adjusted to 5.4-5.8. Distilled water was added to 1000 mL, which was then distributed into 500 mL conical flasks, 250 mL for each. Then, sterilization was performed at 121° C. for 20 min, and cooled down to RT. Ethylene oxide solution was placed in an ice box. 28μL of liquid ethylene oxide was injected into the sterilized medium (containing 100 mg/L of ethylene oxide, which meets the national emission standard) by using a sealed syringe, thus obtaining the enrichment medium A.

3. Preparation of screening purification medium: 40 g of glucose, 5 g of casein trypsin digest, 5 g of pepsin digest of animal tissue, and 15 g of agar were weighed. The pH was adjusted to 5.4. Distilled water was added to 1000 mL, which was distributed into 500 mL conical flasks, 250 mL for each. Then, sterilization was performed at 121° C. for 20 min. When the temperature of the medium was cooled down to 50-56° C., 28μL of liquid ethylene oxide was injected by using a sealed syringe into the sterilized medium to obtain the screening purification medium.

4. Preparation of enrichment medium B: 40 g of glucose, 5 g of casein trypsin digest, 5 g of pepsin digest of animal tissue were weighed. The pH was adjusted to 5.4-5.8. Distilled water was added to 1000 mL, which was distributed into 500 mL conical flasks, 250 mL for each. Then, sterilization was performed at 121° C. for 20 min, and cooled down to room temperature, to obtain the enrichment medium B.

5. 10.0 g of the sample in Step 1 was weighed, 100 mL of 0.03 mol/L phosphate buffer was added. The mixture was stirred and left to stand for 120 min, the large particles of sediment were removed to obtain a suspension. 1 mL of the suspension was added into 10 mL of enrichment medium A, placed on a shaker to perform an oxygen consumption enrichment culture at 37° C. for 24-48 h, with a rotation rate of 200 r/min. Growth situation was observed.

6. Dominant strains in the enrichment medium A were streak-inoculated on screening purification medium to isolate dominant strains for ethylene oxide.

7. The dominant strains for ethylene oxide were selected and cultured in enrichment medium B for 24 h, to obtain the pathogenic bacteria for degrading ethylene oxide, which were preserved in glycerol (the ratio of the culture to 50% glycerol was 1:1) at −80° C.

Three strains of pathogenic bacteria for degrading ethylene oxide were isolated through the above experimental steps.

After 48 h of culturing, one strain was characterized as grey or colorless, opaque, smooth and moist surface, neatly edged, having a colony diameter of about 1.0 mm, and no pigment, and was named as EO-04 original strain.

One strain was characterized as milk white, uneven edged, radial, diameter 3.0-4.0 mm, and no pigment, and was named as EO-07 original strain.

One strain was characterized as grey, opaque, round and smooth, neatly edged, having a colony diameter of 2.0-2.5 mm, no pigment, rod-like body, and with no spores, and was named as EO-10 original strain.

(2) Identification of pathogenic bacteria for degrading ethylene oxide:

Identification contents and methods:

a. Morphological characterization: including observation of colony morphology, microscopic morphology, culture characteristics and Gram staining;

b. Physiological and biochemical characterization: including nutrition type, nitrogen and carbon source utilization capacity, and biochemical tests;

c. Molecular biological characterization: including the procedure of bacterial culture, bacterial DNA extraction, PCR amplification, 16s r DNA sequencing (the DNA in the genome that produces the ribosomal RNA is called the “rRNA gene” or simply “rDNA”) and sequence alignment analysis, wherein the primer pair for PCR amplification was as follows:

Forward primer 27F:
(SEQ ID NO: 1)
5′-AGAGTTTGATCCTGGCTCAG-3′;
Reverse primer 1492R:
(SEQ ID NO: 2)
5′-GGTTACCTTGTTACGACTT-3′.

Results of identification:

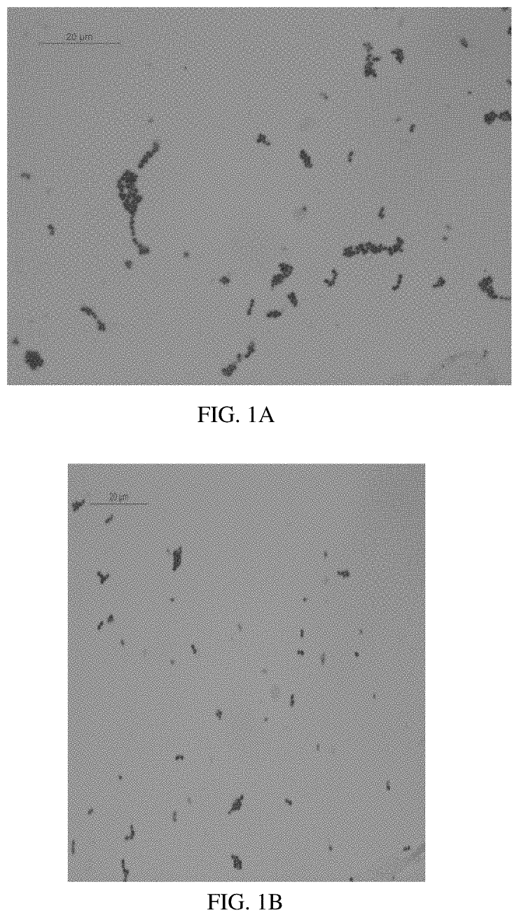

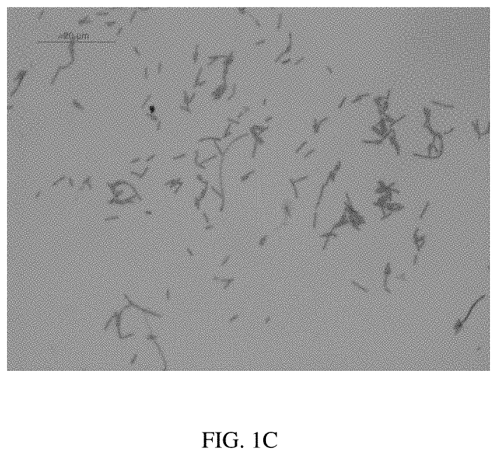

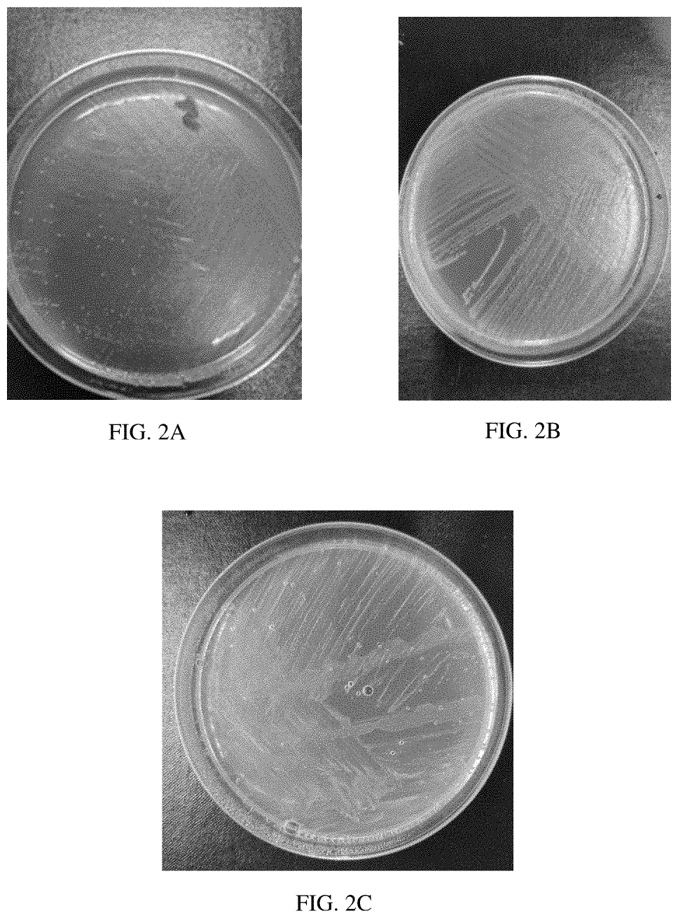

The colonies of EO-04 strain were characterized as gray or colorless, opaque, smooth and moist surface, neatly edged, diameter 0.5-1.0 mm approximately, with no pigment, round or oval body, and with no spores (as shown in FIG. 2A). After Gram staining, the bacteria is purple under microscope (as shown in FIG. 1A). EO-04 is a Gram-positive strain, belongs to facultative anaerobic or aerobic bacteria, has higher nutritional requirements, has good resistance, and can grow well at 30-40° C. and under acidic or alkaline culture condition.

The sequence of 16S rDNA amplified (SEQ ID NO: 3) was subjected to BLAST nucleotide sequence alignment, and was found to have a homology of 99% with the sequence of the 16S rDNA of Enterococcus faecium.

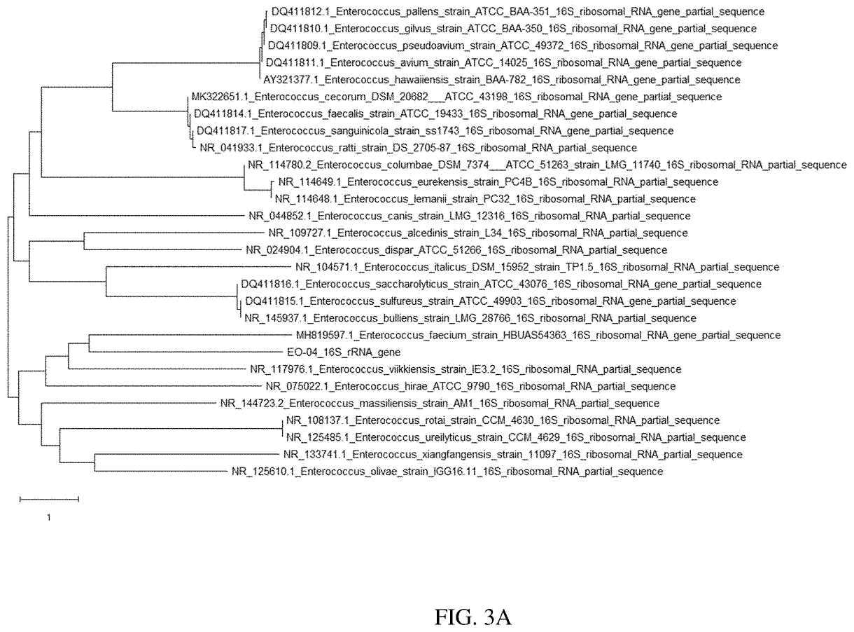

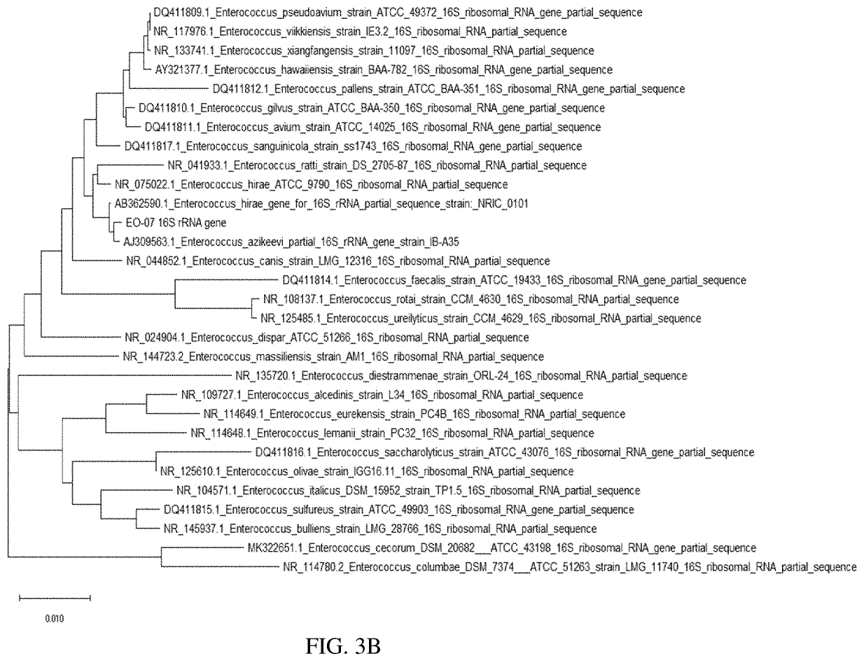

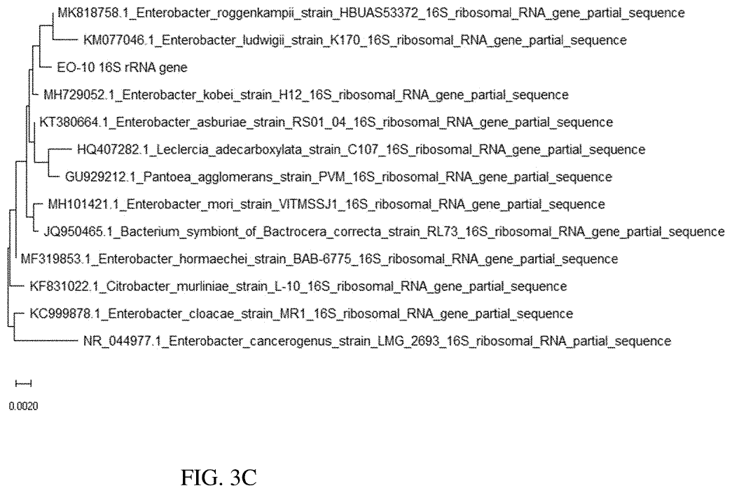

The morphologic identification, physiological and biochemical identification, and molecular biological identification of the EO-04 original strain shows that the EO-04 original strain is Enterococcus faecium, which belongs to Enterococcus bacteria (referring to FIG. 3A).

The morphology of the colonies of the EO-07 original strain shows milk white, uneven edged, diameter 3.0˜4.0 mm, with no pigment, round or oval body, and with no spores (as shown in FIG. 2B). After Gram staining, the bacteria show purple under microscope (as shown in FIG. 1B). EO-07 original strain is a Gram-positive strain, belongs to facultative anaerobic bacteria, saltophilic and alkalophilic, and has an optimum growth temperature of 10-45° C.

The result of gene sequencing was as shown by SEQ ID NO: 4. The 16S rDNA sequence was subjected to BLAST nucleotide sequence alignment, and was found to have a homology of 99% with the sequence of the 16S rDNA of Enterococcus azikeevi, as shown in FIG. 3B.

The morphologic identification, physiological and biochemical identification, and molecular biological identification of the EO-07 original strain show that the EO-07 original strain is Enterococcus azikeevi, which belongs to Enterococcus bacteria.

The morphology of the colonies of the EO-10 original strain shows gray, opaque, round and smooth, neatly edged, having a colony diameter of 2.0-2.5 mm, with no pigment, rod-like body, and with no spores (as shown in FIG. 2C). After Gram staining, the bacteria show red under microscope (as shown in FIG. 1C). EO-10 original strain is a Gram-negative strain, belongs to anaerobic bacteria, pervasive in the natural world, has lower nutritional requirements, easy to culture, fast in reproduction, and suitable for culturing at 30-37° C. under acidic conditions.

The result of sequencing was as shown by SEQ ID NO: 5. The 16S rDNA sequence was subjected to BLAST nucleotide sequence alignment, and was found to have a homology of 99% with the sequence of the 16S rDNA of Enterobacter roggenkampii, as shown in FIG. 3C.

The morphologic identification, physiological and biochemical identification, and molecular biological identification of the EO-10 original strain show that the EO-10 original strain is Enterobacter roggenkampii, which belongs to Enterobacter bacteria.

Inductive Acclimatization of Pathogenic Bacteria for Degrading Ethylene Oxide

(1) Ethylene oxide tolerance inductive acclimatization:

1. Ethylene oxide tolerance acclimatization medium is prepared as follows: 10 g of peptone, 40 g of glucose, and 15 g of agar were weighed. The pH was adjusted to 5.4-5.8. Distilled water was added to 1000 mL and distributed into 250 mL. Sterilization was then performed at 121° C. for 20 min. The medium was heated to melt before use. When the temperature of the medium was cooled down to 50-56° C., 25 mg, 50 mg, 125 mg, and 200 mg of liquid ethylene oxide was added, respectively, by using a sealed syringe, to obtain four ethylene oxide tolerance medium plates containing different concentrations (100 mg/L, 200 mg/L, 500 mg/L, and 800 mg/L, respectively) of ethylene oxide, named as ethylene oxide tolerance acclimatization medium A, ethylene oxide tolerance acclimatization medium B, ethylene oxide tolerance acclimatization medium C, and ethylene oxide tolerance acclimatization medium D.

2. The pathogenic bacteria for degrading ethylene oxide were streak-inoculated on ethylene oxide tolerance acclimatization medium A, and cultured at a constant temperature of 37° C. for 48 h; the single colony having the greatest colony radius was selected for further inoculation on ethylene oxide tolerance acclimatization medium B, and cultured at a constant temperature of 37° C. for 48 h; the single colony having the greatest colony radius was selected for further inoculation on ethylene oxide tolerance acclimatization medium C, and cultured at a constant temperature of 37° C. for 48 h; the single colony having the greatest colony radius was selected for further inoculation on ethylene oxide tolerance acclimatization medium D, and cultured at a constant temperature of 37° C. for 48 h, to obtain ethylene oxide-tolerant bacteria.

(2) Acclimatization for ethylene oxide degradation:

1. Ethylene oxide degradation acclimatization medium is formulated to comprise:

1) 10 g of peptone, 20 g of glucose, and 15 g of agar, pH was adjusted to 5.4-5.8, and distilled water added to 1000 mL.

2) 10 g of peptone, 12 g of glucose, and 15 g of agar, pH adjusted to 5.4-5.8, and distilled water added to 1000 mL.

3) 10 g of peptone, 4 g of glucose, and 15 g of agar, pH adjusted to 5.4-5.8, and distilled water added to 1000 mL.

4) 10 g of peptone, and 15 g of agar, pH adjusted to 5.4-5.8, and distilled water added to 1000 mL.

The medium was distributed into 250 mL. Sterilization was then performed at 121° C. for 20 min. The medium was heated to melt before use. When the temperature of the medium was cooled down to 50-56° C., 200 mg of liquid ethylene oxide was added by using a sealed syringe, to obtain four ethylene oxide degradation acclimatization medium plates containing different contents (50%, 30%, 10%, 0%) of carbon source, named as ethylene oxide degradation acclimatization medium A, ethylene oxide degradation acclimatization medium B, ethylene oxide degradation acclimatization medium C, and ethylene oxide degradation acclimatization medium D.

2. The ethylene oxide-tolerant bacteria were streak-inoculated on ethylene oxide degradation acclimatization medium A, and cultured at a constant temperature of 37° C. for 48 h; the single colony having the greatest colony radius was selected for further inoculation on ethylene oxide degradation acclimatization medium B, and cultured at a constant temperature of 37° C. for 48 h; the single colony having the greatest colony radius was selected for further inoculation on ethylene oxide degradation acclimatization medium C, and cultured at a constant temperature of 37° C. for 48 h; the single colony having the greatest colony radius was selected for further inoculation on ethylene oxide degradation acclimatization medium D, and cultured at a constant temperature of 37° C. for 48 h; the single colony having the greatest colony radius was selected and preserved on an agar medium bevel containing corresponding nutritional components in ethylene oxide degradation acclimatization medium D, to obtain Enterococcus faecium EO-04, Enterococcus azikeevi EO-07, and Enterobacter roggenkampii EO-10, which are capable of degrading ethylene oxide, and preserved under deposit numbers CGMCC No.18434, CGMCC No.18437, and CGMCC No.18440, respectively.

Results for inductive acclimatization of degradation ability for ethylene oxide are shown in Table 1.

TABLE 1
Results for inductive acclimatization of degradation ability for ethylene oxide

| | | | | | | | | |
|---|---|---|---|---|---|---|---|---|
| Carbon source | 100 | 100 | 100 | 100 | 50 | 30 | 10 | 0 |
| content (%) | | | | | | | | |
| EO conc. (mg/L) | 100 | 200 | 500 | 800 | 800 | 800 | 800 | 800 |
| Growth situation | Growing | Growing | Growing | Growing | Growing | Growing | Growing | Growing |
| of EO-04 | | | | | | | | |
| Growth situation | Growing | Growing | Growing | Growing | Growing | Growing | Growing | Growing |
| of EO-07 | | | | | | | | |
| Growth situation | Growing | Growing | Growing | Growing | Growing | Growing | Growing | Growing |
| of EO-10 | | | | | | | | |

As can be seen from Table 1, EO-04 (CGMCC No.18434), EO-07 (CGMCC No.18437), and EO-10 (CGMCC No.18440) strains can all grow normally under conditions that ethylene oxide are used as the only carbon source, and can use ethylene oxide as the carbon source.

The morphologic identification, physiological and biochemical identification, and molecular biological identification of EO-04, EO-07, EO-10 strains performed as mentioned above show that:

EO-04 strain obtained after screening and purification is Enterococcus faecium, which belongs to Enterococcus.

EO-07 strain obtained after screening and purification is Enterococcus azikeevi, which belongs to Enterococcus.

EO-10 strain obtained after screening and purification is Enterobacter roggenkampii, which belongs to Enterobacter.

Identification of the Effect of Ethylene Oxide Degradation by the Strains

1. Preparation of liquid Sabouraud medium: 40 g of glucose, 5 g of casein tryptone, and 5 g of animal tissue pepsin digest were weighed. The pH was adjusted to 5.4-5.8. Distilled water was added to 1000 mL, which was distributed into 500 mL conical flasks, 250 mL for each. Then, sterilization was performed at 121° C. for 20 min, and then was cooled down to RT.

2. Preparation of liquid Sabouraud induction medium: 10 g of peptone was weighed. Distilled water was added to 1000 mL, which was distributed into 400 mL. Then, sterilization was performed at 121° C. for 20 min, and then was cooled down to RT for storage. 160 mg and 320 mg of liquid ethylene oxide was added, respectively, by using a sealed syringe to obtain two liquid Sabouraud induction media containing different concentrations (400 mg/L and 800 mg/L, respectively) of ethylene oxide.

3. Activation: 10μL of the original strains and the conserved strains are inoculated, respectively, in 100 mL of liquid Sabouraud medium, and cultured at 37° C. and 200 rpm for 48 h to a bacteria concentration of 1010-1012 cfu/mL in the culture to obtain activation solutions of the original strains and the conserved strains.

4. Test Group:

Test group 1:5 mL of each of the activation solutions of the conserved strains was inoculated in 400 mL of liquid Sabouraud induction medium.

Test group 2: The original strains was inoculated.

Control group: No inoculation.

5. Incubation was performed at 37° C. in an incubator for 48 h. Degradation was detected by gas chromatography.

Detection was carried out in accordance with GB 15979-2002 of China National Standards as follows:

a series of ethylene oxide standards of 0-200 mg/L concentrations were made by taking a certain volume of pure ethylene oxide gas with a sealed syringe for dissolving in deionized water;

the subject samples to be analyzed were prepared by diluting samples from the treatment and control groups 5 times with deionized water;

after the GC instrument with hydrogen flame detector (FID), is stabilized and under the same conditions, 2μL each of the ethylene oxide standards and the diluted samples to be analyzed were injected into the GC instrument, wherein each sample was measured twice in parallel;

qualitive determination was conducted according to the retention time and quantitative calculation on each peak area was performed to take the average value;

an ethylene oxide standard curve was plotted according to the measurement data of the ethylene oxide standards, and the concentrations of residual ethylene oxide within each sample from the control and treatment groups were found based on the peak area corresponding to ethylene oxide thereof; and Degradation rate was calculated by the following equation:

Degradation rate=(concentration of control group—concentration of test group)/concentration of control group.

Additionally, the percentage of increase in the ethylene oxide degradation ability of the strain before and after acclimation was calculated according to the following formula:

Percentage of increase in degradation ability (%)=(Degradation Rate (%) of the strain after acclimation—Degradation Rate (%) of the strain before acclimation).

Other details of the experiment include Column: Chromosorb 101HP60-80 mesh, glass column 2 m long, diameter 3 mm. Column temperature: 120° C. Detector: 150° C., Gasifier: 150° C.; Carrier gas volume: Nitrogen: 35 ml/min, Hydrogen: 35 ml/min, Air: 350 ml/min, and the pre-column pressure is about 108Kpaa.

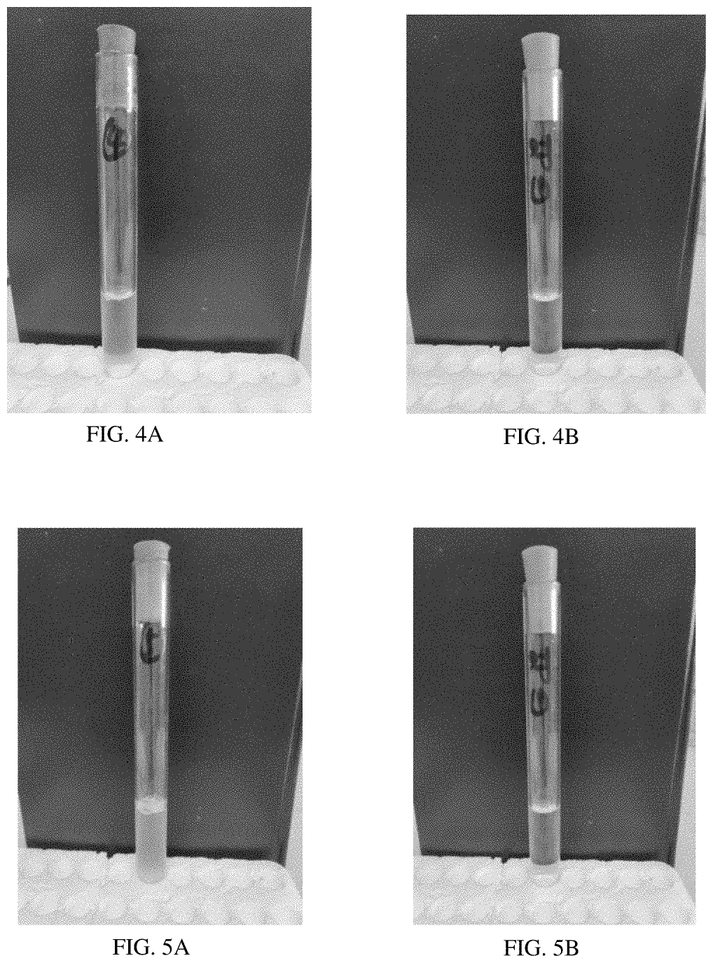

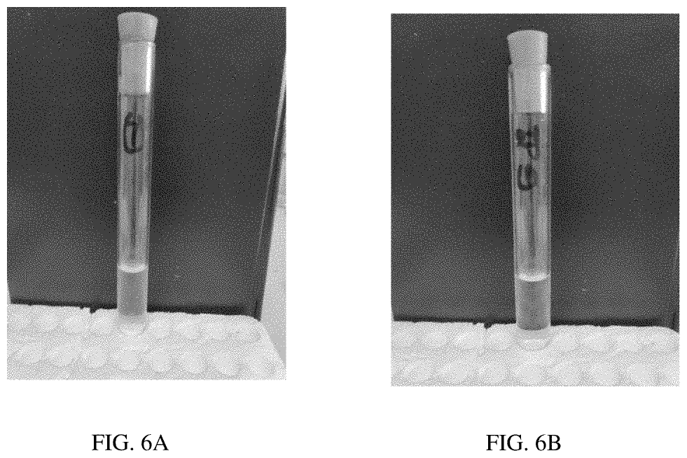

Results are shown in Table 2 and. FIGS. 4A-6B (FIGS. 4A, 5A and 6A show the growth situation of the strains in test group 1 in liquid induction medium, and FIGS. 4B, 5B and 6B show the growth situation of the strains in test group 2 in liquid induction medium).

TABLE 2
Results of ethylene oxide degradation

| | | EO conc. after | EO conc. after | EO conc. of | Degradation | Degradation | Increase in |
|---|---|---|---|---|---|---|---|
| | | degradation of | degradation of | control | rate of | rate of | degradation |
| | | test group 2 | test group 1 | group | test group 2 | test group 1 | capability |
| Strains | EO conc. | (mg/L) | (mg/L) | (mg/L) | (%) | (%) | (%) |
| EO-04 | 800 mg/L | 568.1 | 293.1 | 603.7 | 5.90% | 51.45% | 772.03% |
| | 400 mg/L | 186.1 | 73.2 | 223.6 | 16.77% | 67.26% | 301.07% |
| EO-07 | 800 mg/L | 570.3 | 300.2 | 603.1 | 5.44% | 50.22% | 823.16% |
| | 400 mg/L | 194.8 | 81.1 | 233.5 | 16.57% | 65.27% | 293.90% |
| EO-10 | 800 mg/L | 565.4 | 281.0 | 603.5 | 6.31% | 53.44% | 746.91% |
| | 400 mg/L | 185.1 | 36.8 | 226.5 | 18.28% | 83.75% | 358.15% |

Comparative tests may be carried out in other samples containing ethylene oxide, such as sewage, sludge, exhaust gas, or wastewater, such as industrial (including industries related to petroleum and derivative products), medical treatment (such as ethylene oxide sterilant) and other sewage, sludge, exhaust gas, or wastewater.

An Enterococcus faecium strain bacterium comprising the 16S rDNA sequence of SEQ ID NO: 3; an Enterococcus azikeevi strain bacterium comprising the 16S rDNA sequence of SEQ ID NO: 4; or an Enterobacter roggenkampii strain bacterium comprising the 16S rDNA sequence of SEQ ID NO: 5 can also be used in comparative tests.

Treatment of Ethylene Oxide Sterilization Waste Gas

In general, ethylene oxide sterilization waste gas can be absorbed into water. The water containing the absorbed ethylene oxide can be contacted with an Enterococcus or Enterobacter strain of the present invention in a method of biodegrading ethylene oxide. The water containing the absorbed ethylene oxide can be discharged or transferred to an aerobic or anaerobic vessel, such as an anaerobic sewage tank. A strain of the present invention can then be added to the tank, thereby biodegrading the ethylene oxide.

In particular, 1) after the ethylene oxide sterilizer has sterilized, the ethylene oxide sterilization exhaust gas generated is fed into a hydration system, which uses the internal circulating water to absorb the incoming ethylene oxide sterilization exhaust gas, and several cycles of absorption produce ethylene oxide wastewater containing about 122.62 mg/L of ethylene oxide.

(2) The concentration of about 122.62 mg/L of ethylene oxide was passed into a mixed ethylene oxide treatment cell inoculated with EO-04, EO-07, and EO-10 strains, and the total strain concentration was 10¹⁰10¹² cfu/mL, and the inoculation amount was 1%-2%, the strain(s) used the active sludge in the anaerobic ethylene oxide treatment cell as the culture, ethylene oxide was used as the carbon source and energy for metabolism, growth and proliferation, thus achieving the purpose of ethylene oxide treatment. The mixture in the treatment cell was continuously stirred, the temperature was controlled at 32° C-42° C. and the treatment was for 48 hours.

The results showed that the residual concentration of ethylene oxide in the treated wastewater was 19.87 mg/L with a treatment efficiency of 83.80%.

The above concentrations were detected by gas chromatography in accordance with GB 15979-2002 (Appendix D), which is explained above. The degradation rate was calculated according to the following formula: Degradation rate =(starting concentration—residual concentration)/starting concentration×100.

As another practical application, activated sludge can be contacted with a strain of the present invention, thereby biodegrading ethylene oxide in the activated sludge.

In the above-described tests and applications, the degradation rate is at least 10%, 15%, 20%, 25%, 30%, 35%, 40%, 50%, 60%, 70%, 75%, 80%, 90%, 95%, 100%, 125%, 150%, 200%, or 500% greater relative to the degradation rate of ethylene oxide in the absence of a bacterial strain of the invention.

Each embodiment is described in this specification in a progressive manner, with each embodiment highlighting the differences from the other embodiments, and the same similarities between the embodiments can be found in each other's descriptions.

The foregoing description of the disclosed embodiments enables a person skilled in the field to implement or use the present invention. Various modifications of these embodiments will be apparent to a person of skill in the art in this field, and the general principles defined herein may be implemented in other embodiments without departing from the spirit or scope of the present invention. Accordingly, the present invention will not be limited to these embodiments as shown herein, but will conform to the broadest possible scope consistent with the principles and novel features disclosed herein.

1. A product selected from the group consisting of:
a pathogenic bacteria for degrading ethylene oxide which is Enterococcus faecium EO-04, with the deposit number CGMCC No.18434;
a pathogenic bacteria for degrading ethylene oxide which is Enterococcus azikeevi EO-07, with the deposit number CGMCC No.18437;
a pathogenic bacteria for degrading ethylene oxide which is Enterobacter roggenkampii EO-10, with the deposit number CGMCC No.18440; and
a degradation agent for degrading ethylene oxide, comprising one or more strains selected from the group consisting of an Enterococcus faecium EO-04, with the deposit number CGMCC No.18434; an Enterococcus azikeevi EO-07, with the deposit number CGMCC No.18437; and an Enterobacter roggenkampii EO-10, with the deposit number CGMCC No.18440.

2. The product according to claim 1, wherein the degradation agent is prepared by culturing the one or more strains.

3. The product according to claim 1, wherein a final concentration of the one or more strains in the degradation agent is at least 10¹⁰ cfu/mL.

4. A method for preparing a degradation agent for degrading ethylene oxide, wherein the method is selected from the group consisting of:
i) incubating one or more strains selected from the group consisting of an Enterococcus faecium EO-04, with the deposit number CGMCC No.18434; an Enterococcus azikeevi EO-07, with the deposit number CGMCC No.18437; and an Enterobacter roggenkampii EO-10, with the deposit number CGMCC No.18440 in a liquid Sabouraud medium and at a temperature of 20-40° C.; and
ii) a)inducted acclimation for ethylene oxide tolerance, comprising: successively passaging the bacteria with ethylene oxide degradation potential by streaking the same on a series of acclimation medium for ethylene oxide tolerance containing a gradient of increasing ethylene oxide concentrations from 100 to 800 mg/L; after each passaging, incubating at 20-40° C. for 24 to 48 hours, and selecting a single colony with a largest radius for next passaging; and finally selecting a single colony with a largest colony radius on an acclimation medium containing ethylene oxide of 500-800 mg/L to obtain a bacteria strain of ethylene oxide tolerance; and
b) inducted acclimation for ethylene oxide degradation ability, comprising: successively passaging the bacteria strain of ethylene oxide tolerance by steaking the same on a series of acclimation medium for ethylene oxide degradation containing ethylene oxide of 500-800 mg/L and a gradient of decreasing proportion of carbon source from 50% to 0%; after each passaging, incubating at 20-40° C. for 24 to 48 hours, and selecting a single colony with a largest radius for next passaging; and finally selecting a single colony with a largest colony radius on the acclimation medium containing 500-800 mg/L of ethylene oxide and 0% of carbon source to obtain the bacteria strain having ethylene oxide tolerance and degradation ability;
wherein the series of acclimation medium for ethylene oxide tolerance have ethylene oxide concentrations increasing between 100 and 800 mg/L and comprises, by mass, 10 parts of peptone, 40 parts of glucose, and 15 parts of agar, which are mixed with water, adjusted to a pH of 5.4-5.8, and the volume brought to 1000 parts with water;
and wherein the series of acclimation medium for ethylene oxide degradation have an ethylene oxide concentration of 500-800 mg/L and comprises, by mass, 10 parts of peptone, glucose decreasing from 20 parts to 0 parts, and agar 15 parts, which are mixed with water, adjusted to a pH of 5.4-5.8, and the volume brought to 1000 parts with water.

5. The method according to claim 4, wherein the liquid Sabouraud medium comprises: by mass, 40 parts of glucose, 5 parts of casein trypsin digest, and 5 parts of animal tissue pepsin digest, which are combined in water, adjusted to a pH of 5.4-5.8, and the volume brought to 1000 parts with water.

6. A method for biodegrading ethylene oxide or decreasing the amount of ethylene oxide in sample, the method selected from the group consisting of:
i) degrading ethylene oxide with one or more strains selected from the group consisting of an Enterococcus faecium EO-04, with the deposit number CGMCC No.18434; an Enterococcus azikeevi EO-07, with the deposit number CGMCC No.18437; and an Enterobacter roggenkampii EO-10, with the deposit number CGMCC No.18440; and
ii) a) adding to a sample comprising ethylene oxide an amount a pure culture of an Enterococcus faecium, Enterococcus azikeevi, or Enterobacter roggenkampii strain bacterium, and
b) allowing the bacterium to degrade the ethylene oxide, thereby decreasing the amount of ethylene oxide,
wherein the 16S rDNA sequence of the Enterococcus faecium strain bacterium is SEQ ID NO. 3; the 16S rDNA sequence of the Enterococcus azikeevi strain bacterium is SEQ ID NO. 4; or the 16S rDNA sequence of the Enterobacter roggenkampii strain bacterium is SEQ ID NO. 5.

7. The method according to claim 6, wherein the method is used to degrade ethylene oxide in waste gas or waste water and comprises mixing the waste gas or waste water with one or more strains selected from the group consisting of an Enterococcus faecium EO-04, with the deposit number CGMCC No.18434; an Enterococcus azikeevi EO-07, with the deposit number CGMCC No.18437; and an Enterobacter roggenkampii EO-10, with the deposit number CGMCC No.18440.

8. The method of claim 6, wherein the degrading ethylene oxide with one or more strains selected from the group consisting of an Enterococcus faecium EO-04, with the deposit number CGMCC No.18434; an Enterococcus azikeevi EO-07, with the deposit number CGMCC No.18437; and an Enterobacter roggenkampii EO-10, with the deposit number CGMCC No.18440 comprises:
incubating the one or more strains in a liquid Sabouraud medium and at a temperature of 20-40° C.

9. The method according to claim 6, wherein the degradation rate is at least 10% greater relative to the degradation rate of ethylene oxide in the absence of the strain for degrading ethylene oxide.

10. The method according to claim 6, wherein the concentration of the strain for degrading ethylene oxide ranges from 10¹⁰ cfu/mL to 10¹² cfu/mL.

11. The method according to claim 6, wherein the Enterococcus faecium, Enterococcus azikeevi, or Enterobacter roggenkampii strain bacterium is capable of using ethylene oxide as a carbon source and is capable of growing normally with ethylene oxide as the sole carbon source in the culture.

12. The method according to claim 6, wherein the Enterococcus faecium strain bacterium is Enterococcus faecium EO-04, with the deposit number CGMCC No.18434; the Enterococcus azikeevi strain bacterium is Enterococcus azikeevi EO-07, with the deposit number CGMCC No.18437; and the Enterobacter roggenkampii strain bacterium is Enterobacter roggenkampii EO-10, with the deposit number CGMCC No.18440.
